(19) United States
(12) Patent Application Publication
(10) Pub. No.: US 2008/0038217 A1
(43) Pub. Date: Feb. 14, 2008

(54) COSMETIC COMPOSITION COMPRISING AT LEAST ONE SILICONE/ACRYLATE COPOLYMER AND AT LEAST ONE NONIONIC POLYMER COMPRISING AT LEAST ONE VINYLLACTAM UNIT

(75) Inventor: Christine Dupuis, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal.

(21) Appl. No.: 11/837,882

(22) Filed: Aug. 13, 2007

Related U.S. Application Data
(63) Continuation of application No. 09/663,183, filed on Sep. 15, 2000, now abandoned.

(30) Foreign Application Priority Data
Sep. 16, 1999 (FR) 99 11591

(51) Int. Cl. A61K 8/72; A61Q 19/00; A61Q 5/00

(52) U.S. Cl. 424/70.12; 424/70.16; 424/78.03

(57) ABSTRACT

The present invention relates to cosmetic compositions comprising at least one specific silicone/acrylate copolymer and at least one nonionic polymer comprising at least one vinyllactam unit. The present invention is also directed to a processes, in particular a process for fixing and/or holding the hairstyle comprising the in use of said composition, as well as to the use of this composition for the manufacture of a cosmetic formulation intended in particular for holding and/or shaping the hairstyle.

The present invention relates to cosmetic compositions comprising at least one silicone/acrylate copolymer and at least one nonionic polymer comprising at least one vinyllactam unit. The present invention also relates to cosmetic processes, in particular processes for fixing and/or holding the hairstyle by using the inventive composition, as well as to processes for making cosmetic products comprising including in the products at least one composition according to the present invention.

Among hair products intended for shaping and/or holding the hairstyle which are commercially available are spray compositions which essentially consist of a solution, usually an alcoholic or aqueous solution, and one or more materials. Generally, these materials are polymeric resins. The function of these resins is to form welds between the hairs, and therefore, these materials are also known as fixing materials. These fixing materials may be mixed with various cosmetic adjuvants. The compositions comprising these fixing materials are generally packaged either in a suitable aerosol container placed under pressure with the aid of a propellant or in a pump-dispenser bottle.

Hair gels and hair mousses which are generally applied to wet hair are also known. Drying or blow-drying of the hair may be carried out after these products have been applied to the wet hair in order to shape and fix the hairstyle. As with the aforementioned sprays, these gels and mousses may also contain polymeric resins.

However, these hair compositions may adversely affect the cosmetic properties of the hair. That is, the hair may become coarse and difficult to disentangle, it may lose its pleasant feel and appearance or it may lack body. Styling compositions are thus sought which afford good cosmetic properties, in particular in terms of ease of disentangling and softness and feel of the hair.

In addition, these hair compositions may also have the major drawback of giving rise to a powdering effect. As used herein, the term “powdering” means the aptitude of the composition, upon drying after application to the hair, to form a powder.

Needless to say, because the powder falls on the user's shoulders and clothing or attaches to a comb or brush, this powdering effect can be a major drawback of such compositions.

Thus, there is a need for cosmetic compositions, in particular for styling the hair, which do not have one or all of the aforementioned disadvantages and, in particular, which fix the hairstyle well while affording good cosmetic properties and which do not give rise to a powdering effect.

The inventor has discovered, surprisingly and unexpectedly, that when specific silicone/acrylate copolymers are combined with certain specific polymers, it is possible to obtain cosmetic compositions which may display at least one of the desired qualities expressed above.

Specifically, one subject of the present invention is cosmetic compositions comprising at least one silicone/acrylate copolymer and at least one nonionic polymer comprising at least one vinyllactam unit, wherein the at least one silicone/acrylate copolymer is derived from radical-mediated polymerization of at least one ethylenically unsaturated monomer (a) and at least one silicone derivative (b) comprising at least one oxyalkylene group. These compositions may further comprise at least one cosmetically acceptable medium.

The present invention is also directed to processes, in particular processes for fixing and/or holding the hairstyle, using the inventive compositions.

Another subject of the present invention is processes of making a cosmetic product comprising including in said product at least one composition according to the present invention. This cosmetic product may be a hair product, a product for the skin, a product for the nails, a product for the lips, a product for the eyebrows and/or a product for the eyelashes.

The silicone/acrylate copolymers suitable for use in the present invention can be chosen from those described in International patent application WO 99/04750, the disclosure of which is incorporated by reference. For example, the copolymer sold by BASF under the name Luviflex Silk may be used. This copolymer is a grafted copolymer derived from monomers derived from tert-butyl acrylate/methacrylic acid and silicone copolyol.

For example, the at least one ethylenically unsaturated monomer (a) can be chosen from at least one monomer of formula (Iₐ): 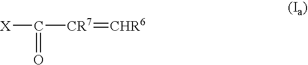
in which:

X is chosen from OH, OM, OR^{a}, NH₂, NHR⁸ and N(R⁸)₂ wherein:
R⁸, which may be identical or different, are each chosen from hydrogen atoms, linear and branched C₁ to C₄₀ alkyl groups, optionally substituted with at least one group chosen from alkoxy groups, amino groups and carboxyl groups, monohydroxylated linear and branched C₁ to C₄₀ alkyl groups, optionally substituted with at least one group chosen from alkoxy groups, amino groups and carboxyl groups, polyhydroxylated linear and branched C₁ to C₄₀ alkyl groups, optionally substituted with at least one group chosen from alkoxy groups, amino groups and carboxyl groups, hydroxylated polyethers; and
M is chosen from Na⁺, K⁺, Mg⁺⁺, NH⁴⁺, an alkylammonium group, a dialkylammonium group, a trialkylammonium group and a tetraalkylammonium group; and

R⁷ and R⁶, which may be identical or different, are each chosen from hydrogen atoms, linear and branched C₁ to C₈ alkyl groups, methoxy groups, ethoxy groups, 2-hydroxyethoxy groups, 2-methoxyethoxy groups, 2-ethoxyethyl groups, CN groups, COOH groups and COOM groups, wherein M is defined as above.

For example R⁸, which may be identical or different, can each be chosen from N,N-dimethylaminoethyl groups, 2-hydroxyethyl groups, 2-methoxyethyl groups, 2-ethoxyethyl groups, hydroxypropyl groups, methoxypropyl groups and ethoxypropyl groups.

In the present invention, the at least one monomer of formula (Ia) can be chosen from acrylic acid and its salts, esters and amides. The at least one monomer of formula (Ia) may also optionally be substituted. For example, the at least one monomer of formula (Ia) can also be chosen from methacrylic acid, ethacrylic acid and 3-cyanoacrylic acid.

The at least one monomer of formula (Ia) can also be chosen from esters which may be chosen from derivatives of linear C₁ to C₄₀ alkyls, derivatives of branched C₃ to C₄₀ alkyls, derivatives of C₃ to C₄₀ carboxylic alcohols, derivatives of polyfunctional alcohols comprising 2 to 8 hydroxyl groups, derivatives of alcohol ethers and derivatives of polyalkylene glycols. Non-limiting examples of polyfunctional alcohols comprising 2 to 8 hydroxyl groups which may be used according to the present invention include ethylene glycol, hexylene glycol, glycerol and 1,2,6-hexanetriol. Non-limiting examples of alcohol ethers which may be used according to the present invention include methoxymethanol and ethoxyethanol.

The at least one monomer of formula (Ia) can also be chosen from N,N-dialkylaminoalkyl acrylates, N,N-dialkylaminoalkyl methacrylates, N-dialkylaminoalkyl acrylamides and N-dialkylaminoalkyl methacrylamides, wherein the amide group may optionally be unsubstituted, N-alkyl-monosubstituted, N-alkylamino-monosubstituted or N,N-dialkylamino-disubstituted, and wherein the alkyl and alkylamino groups are chosen from groups derived from linear C₁ to C₄₀ carboxylic units and groups derived from branched C₃ to C₄₀ carboxylic units.

The at least one ethylenically unsaturated monomer (a) can be chosen from C₁ to C₄₀ vinyl esters, C₁ to C₄₀ alkyl esters, linear C₃ to C₄₀ carboxylic acids, branched C₃ to C₄₀ carboxylic acids, vinyl halides, allyl halides, vinyllactams, heterocyclic compounds substituted with at least one group chosen from vinyl groups and allyl groups, N-vinylimidazoles, diallylamines, vinylidene chloride, carbon-based unsaturated compounds, acrylic acid derivatives quaternized with epichlorohydrin and methacrylic acid derivatives quaternized with epichlorohydrin. Non-limiting examples of vinyllactams include vinylpyrrolidone and vinylcaprolactam. Non-limiting examples of heterocyclic compounds substituted with at least one group chosen from vinyl groups and allyl groups include vinylpyridine, vinyloxazoline and allylpyridine. Non-limiting examples of carbon-based unsaturated compounds include styrene and isoprene.

In one embodiment, the at least one ethylenically unsaturated monomer (a) is chosen from N-vinylimidazoles, diallylamines, vinylidene chloride, carbon-based unsaturated compounds, acrylic acid derivatives quaternized with epichlorohydrin and methacrylic acid derivatives quaternized with epichlorohydrin.

Representative at least one ethylenically unsaturated monomers (a) according to the present invention comprise acrylic acid, methacrylic acid, ethacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate, n-butyl methacrylate, isobutyl methacrylate, t-butyl methacrylate, 2-ethylhexyl methacrylate, decyl methacrylate, methyl ethacrylate, ethyl ethacrylate, propyl ethacrylate, n-butyl ethacrylate, isobutyl ethacrylate, t-butyl ethacrylate, 2-ethylhexyl ethacrylate, decyl ethacrylate, 2,3-hydroxypropyl acrylate, 2,3-dihydroxypropyl methacrylate, 2-dihydroxyethyl acrylate, hydroxypropyl acrylate, 2-hydroxyethyl methacrylate, 2-hydroxyethyl ethacrylate, 2-methoxyethyl acrylate, 2-ethoxyethyl methacrylate, 2-ethoxyethyl ethacrylate, hydroxypropyl methacrylate, glyceryl monoacrylate, glyceryl monomethacrylate, polyalkylene glycol (meth)acrylates, unsaturated sulphonic acids, acrylamide, methacrylamide, ethacrylamide, N,N-dimethylacrylamide, N-ethylacrylamide, N-ethylmethacrylamide, 1-vinyl-imidazole, N,N-dimethylaminoethyl (meth)acrylate, maleic acid, fumaric acid, maleic anhydride, monoesters of maleic anhydride, crotonic acid, itaconic acid, vinyl ethers, vinylformamide, vinylamine, vinylpyridine, vinylimidazole, vinylfuran, styrene, styryl sulphonate and allyl alcohol.

According to the present invention, the at least one ethylenically unsaturated monomer (a) can also comprise at least one entity chosen from silicon atoms, fluorine atoms and thio groups.

If the at least one ethylenically unsaturated monomer (a) comprises at least one acid group, the at least one acid group can be neutralized before or after polymerization, and partially or totally, such that the solubility and/or the degree of dispersion in water is adjusted to the desired level. Non-limiting examples of agents which may be used for the neutralization according to the present invention include mineral bases and organic bases. For example, the mineral base can be chosen from sodium carbonate. Representative organic bases are amino alcohols and diamines. For example, amino alcohols can be chosen from alkanolamines and these alkanolamines can be chosen from methanolamine, 2-amino-2-methyl-1-propanol and triethanolamine. A representative diamine is lysine.

Further, if the at least one ethylenically unsaturated monomer (a) comprises at least one basic nitrogen atom, the at least one basic nitrogen atom can be quaternized. Similarly, if the at least one ethylenically unsaturated monomer (a) comprises at least two ethylenic double bonds, the at least one ethylenically unsaturated monomer (a) can be at least partially crosslinked.

In the present invention, the at least one silicone derivative (b) suitable for use in the present invention can be chosen from compounds known under the INCI names as dimethicone copolyols and silicone surfactants. For example, the compounds sold under the brand names Abil® by Goldschmidt, Alkasil® by Rhone-Poulenc, silicone Polyol Copolymer® by Genesee, Besil by Wacker, Silwet® by OSI and Dow Corning 190® by Dow Corning may be used.

In one embodiment, the at least one silicone derivative (b) is chosen from at least one derivative of formula I: 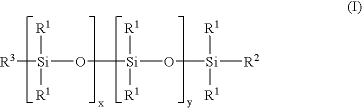
in which:

x and y, which may be identical or different, are each chosen from integers such that the number average molecular weight of said at least one silicone/acrylate copolymer ranges from 300 to 30,000;

R² and R³, which may be identical or different, are each chosen from CH₃ and groups of formula: 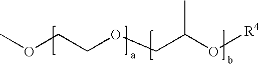
in which:
   R⁴ is chosen from hydrogen, CH₃, groups of formula: 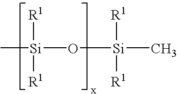
      in which:
x is an integer chosen such that the number average molecular weight of said at least one silicone/acrylate copolymer ranges from 300 to 30,000; and
R¹, which may be identical or different, are each chosen from C₁ to C₂₀ aliphatic hydrocarbons, C₃ to C₂₀ aromatic groups, C₃ to C₂₀ cycloaliphatic hydrocarbons, groups comprising both aromatic groups and aliphatic groups and groups of formula: 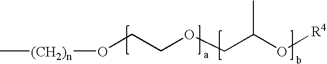
   in which:
n is an integer ranging from 1 to 6;
a and b, which may be identical or different, are each chosen from integers ranging from 0 to 50; and
and R⁴ is defined as above;
and groups of formula: 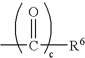
   in which:
c is equal to 0 or 1; and
R⁶ is chosen from a C₁ to C₄₀ group, optionally comprising at least one group chosen from amino groups, carboxyl groups and sulfonyl groups, and,
if c is equal to zero, R₆ is chosen from an anion of an inorganic acid; and

R¹, which may be identical or different, are each chosen from C₁ to C₂₀ aliphatic hydrocarbons, C₃ to C₂₀ aromatic groups, C₃ to C₂₀ cycloaliphatic hydrocarbons, groups comprising both aromatic groups and aliphatic groups, and groups of formula: 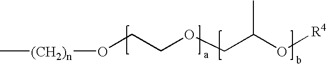
in which:
n is an integer ranging from 1 to 6;
a and b, which may be identical or different, are each chosen from integers ranging from 0 to 50; and R⁴ is defined as above;
   with the proviso that said at least one silicone derivative (b) comprises at least one oxyalkylene group.

In the present invention, R¹, which may be identical or different, can be chosen from methyl groups, ethyl groups, propyl groups, butyl groups, isobutyl groups, pentyl groups, isopentyl groups, hexyl groups, octyl groups, decyl groups, dodecyl groups, octadecyl groups, cycloaliphatic groups, aromatic groups and groups comprising both aromatic and aliphatic groups. For example, cycloaliphatic groups can be chosen from cyclohexyl groups. Representative aromatic groups comprise phenyl groups and naphthyl groups. Representative groups comprising both aromatic and aliphatic groups comprise benzyl groups, phenylethyl groups, tolyl groups and xylyl groups.

In the present invention, R⁴ can be chosen from groups of formula —(CO)_{c}—R⁶, wherein when c is equal to 1, R⁶ can be chosen from a group comprising from 1 to 40 carbon atoms, optionally additionally comprising at least one group chosen from NH₂ groups, COOH groups and SO₃H groups, wherein said group comprising 1 to 40 carbon atoms can be chosen from an alkyl group, a cycloalkyl group and an aryl group. In one embodiment, R⁴ is chosen from groups of formula —(CO)ₙ—R⁶, wherein c is equal to zero and R⁶ is chosen from phosphates and sulphates.

For example, the at least one silicone derivative (b) can be chosen from at least one derivative of formula: 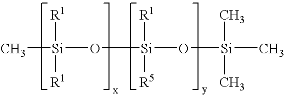
wherein R₁, x and y are as previously described and R⁵, which may be identical or different, can each be chosen from groups of formula: 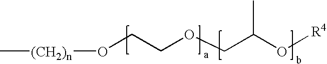
in which n can be an integer ranging from 1 to 6, a and b, which may be identical or different, can each be chosen from integers ranging from 0 to 50 and R⁴ is as defined above, with the proviso that said at least one silicone derivative (b) comprises at least one oxyalkylene unit.

According to the present invention, the at least one silicone derivative (b) may be present in a proportion generally ranging from 0.1% to 50% by weight relative to the total weight of the at least one silicone/acrylate copolymer. For example, the at least one silicone derivative (b) may be present in a proportion ranging from 1% to 20% by weight relative to the total weight of the at least one silicone/acrylate copolymer.

The at least one silicone/acrylate copolymer can be chosen from, for example, water-soluble silicone/acrylate copolymers and silicone/acrylate copolymers having a dispersibility in water is such that, in a water/ethanol mixture dosed at 50/50 by volume, they are soluble in a proportion of generally greater than 0.1 g/l, such as greater than 0.2 g/l.

The composition of the present invention can generally comprise from 0.1% to 20% of the at least one silicone/acrylate copolymer relative to the total weight of the composition, such as from 0.5% to 10%.

According to the present invention, the at least one nonionic polymer comprising at least one vinyllactam unit can be chosen from at least one nonionic polymer comprising at least one unit of formula:

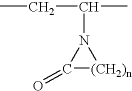

in which n is an integer chosen from 3, 4 and 5.

The at least one nonionic polymer comprising at least one vinyllactam unit can also be chosen from copolymers which further comprise at least one unit of formula: 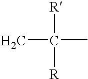
in which:
R is chosen from an acyloxy group wherein the alkoxy group of the carbalkoxy group may optionally be substituted with at least one group which may optionally be substituted with at least one alkyl group, wherein said at least one group can be chosen from hydroxyl groups and aryl groups; and

R′ is chosen from a hydrogen atom and an alkyl group.

For example, R can be chosen from an acyloxy group wherein the alkoxy group of the carbalkoxy group may optionally be substituted with at least one phenyl group.

As used herein, the term “alkyl,” unless defined otherwise, means C₁-C₁₀ alkyl groups chosen from linear and branched C₁-C₁₀ alkyl groups. For example, the alkyl groups can be chosen from C₁-C₄ alkyl groups. Non-limiting examples of C₁-C₄ alkyl groups include methyl groups, ethyl groups, n-propyl groups, isopropyl groups, n-butyl groups and t-butyl groups.

As used herein, the term “acyl,” unless defined otherwise, means acyl groups in which the alkyl group is chosen from linear and branched C₁-C₁₀ alkyls. For example, acyl groups can be chosen from acetyl groups and propionyl groups.

The at least one non-ionic polymer comprising at least one vinyllactam unit according to the invention can be chosen from those described in U.S. Pat. Nos. 3,770,683, 3,929,735, 4,521,504, 5,158,762 and 5,506,315 and in patent applications WO 94/121148, WO 96/06592 and WO 96/10593, the disclosures of which are incorporated by reference. These polymers may be in pulverulent form or in the form of a solution or suspension.

The at least one non-ionic polymer comprising at least one vinyllactam unit can be chosen from, for example, polyvinylpyrrolidones, polyvinylcaprolactams, polyvinylpyrrolidone/vinyl acetate copolymers and polyvinylpyrrolidone/vinyl acetate/vinyl propionate terpolymers (sold in particular under the name Luviskol VAP 343 by the company BASF).

The number-average molecular mass of the at least one non-ionic polymer comprising at least one vinyllactam unit may be greater than about 5000. For example, the number-average molecular mass may range from about 10,000 to about 1,000,000 or from about 10,000 to about 100,000.

In the present invention, the at least one non-ionic polymer comprising at least one vinyllactam unit may be used in dissolved form or in the form of dispersions of solid polymer particles.

The inventive composition can comprise from 0.1% to 10% of the at least one nonionic polymer comprising at least one vinyllactam unit relative to the total weight of the at least one silicone/acrylate copolymer, such as from 0.2% to 5%.

As previously mentioned, the compositions according to the present invention may further comprise at least one cosmetically acceptable medium. The at least one cosmetically acceptable medium can be chosen from water and cosmetically acceptable solvents. The cosmetically acceptable solvents can be chosen from alcohols and mixtures comprising water and at least one solvent. For example, the solvents can be chosen from C₁-C₄ alcohols. Non-limiting examples of C₁-C₄ alcohols which may be used according to the present invention include ethanol and isopropanol. In one embodiment, the C₁-C₄ alcohol is ethanol.

According to the present invention, the composition can further comprise at least one suitable additive chosen from anionic surfactants, cationic surfactants, nonionic surfactants, amphoteric surfactants, fragrances, screening agents, preserving agents, proteins, vitamins, polymers different from the at least one silicone/acrylate copolymer and different from the at least one nonionic polymer defined herein, plant oils, mineral oils, synthetic oils and any other additive conventionally used in cosmetic compositions.

Needless to say, a person skilled in the art will take care to select the at least one suitable additive such that the advantageous properties of the composition in accordance with the invention are not, or are not substantially, adversely affected by the addition(s) envisaged.

These compositions may be packaged in various forms, for example, in pump-dispenser bottles or in aerosol containers, in order to ensure application of the composition in vaporized form or in the form of a mousse. Such packaging forms may be used, for example, when it is desired to obtain a spray, a lacquer or a mousse for fixing or treating hair. The compositions in accordance with the invention can also be in the form of creams, gels, emulsions, lotions or waxes.

When the composition according to the invention is packaged in aerosol form in order to obtain a lacquer or a mousse, it may comprise at least one propellant which may be chosen from volatile hydrocarbons such as n-butane, propane, isobutane and pentane, a halogenated hydrocarbon and mixtures thereof. Carbon dioxide, nitrous oxide, dimethyl ether (DME), nitrogen or compressed air can also be used as the at least one propellant.

The at least one propellant may be generally present in a concentration ranging from 5% to 90% by weight relative to the total weight of the composition in the aerosol device, such as from 10% to 60%.

The compositions in accordance with the invention can be applied to the skin, the nails, the lips, the hair, the eyebrows and/or the eyelashes.

The compositions in accordance with the invention may be suitable for dry or wet hair, and may, for example, be applied as styling products.

The invention will be illustrated more fully with the aid of the following non-limiting example. As used herein, “A.M.” means active material. As used herein, “PDMS” means polydimethyl silicone. As used herein, “AMP” means 2-amino-2-methyl-1-propanol.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result. The percentages are given on a weight basis.

### EXAMPLE

The following composition was prepared in a pump-dispenser bottle:

| | |
|---|---|
| Luviflex Silk (BASF)* | 3% A.M. |
| Luviskol plus** | 2% A.M. |
| AMP | qs pH = 7 |
| Ethanol | qs 100 g |

*silicone/acrylate copolymer (t-butyl-acrylate/methacrylic acid/PDMS polyether)
**polyvinylcaprolactam

This composition was applied by spraying it onto a lock of European chestnut-brown hair. Good hold and very good cosmetic qualities were obtained.

1. A composition comprising:
(a) at least one silicone/acrylate copolymer; and
(b) at least one nonionic polymer comprising at least one vinyllactam unit chosen from polyvinylpyrrolidone/vinyl acetate/vinyl proprionate terpolymers,
wherein said at least one silicone/acrylate copolymer is derived from radical-mediated polymerization of:
(i) at least one ethylenically unsaturated monomer (a); and
(ii) at least one silicone compound (b) comprising at least one oxyalkylene group.

2. A composition according to claim 1 further comprising a cosmetically acceptable medium.

3. A composition according to claim 1, wherein said at least one ethylenically unsaturated monomer (a) is chosen from at least one monomer of formula (Ia): 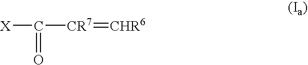
in which:
X is chosen from OH, OM, OR⁸, NH₂, NHR⁸ and N(R⁸)₂, wherein:
R⁸, which may be identical or different, are each chosen from hydrogen atoms, linear and branched C₁ to C₄₀ alkyl groups, optionally substituted with at least one group chosen from alkoxy groups, amino groups and carboxyl groups, monohydroxylated linear and branched C₁ to C₄₀ alkyl groups, optionally substituted with at least one group chosen from alkoxy groups, amino groups and carboxyl groups, polyhydroxylated linear and branched C₁ to C₄₀ alkyl groups, optionally substituted with at least one group chosen from alkoxy groups, amino groups and carboxyl groups, and hydroxylated polyethers; and
M is chosen from Na⁺, K⁺, Mg⁺, NH⁴⁺, an alkylammonium group, a dialkylammonium group, a trialkylammonium group and a tetraalkylammonium group;
R⁶ and R⁷, which may be identical or different, are each chosen from hydrogen atoms, linear and branched C1 to C8 alkyl groups, methoxy groups, ethoxy groups, 2-hydroxyethoxy groups, 2-methoxyethoxy groups, 2-ethoxyethyl groups, CN groups, COOH groups and COOM groups, wherein M is defined as above.

4. A composition according to claim 3, wherein said linear and branched C₁ to C₄₀ alkyl groups optionally substituted with at least one group chosen from alkoxy groups, amino groups and carboxyl groups are each chosen from N,N-dimethylaminoethyl groups, 2-methoxyethyl groups, 2-ethoxyethyl groups, methoxypropyl groups and ethoxypropyl groups.

5. A composition according to claim 3, wherein said monohydroxylated linear and branched C₁ to C₄₀ alkyl groups optionally substituted with at least one group chosen from alkoxy groups, amino groups and carboxyl groups are each chosen from 2-hydroxyethyl groups and hydroxypropyl groups.

6. A composition according to claim 3, wherein said at least one monomer of formula (Ia) is chosen from acrylic acid, the salts of acrylic acid, esters and amides.

7. A composition according to claim 3, wherein said at least one monomer of formula (Ia) is chosen from methacrylic acid, ethacrylic acid and 3-cyanoacrylic acid.

8. A composition according to claim 3, wherein said at least one monomer of formula (Ia) is chosen from esters obtained from linear C₁ to C₄₀ alkyls, branched C₃ to C₄₀ alkyls, C₃ to C₄₀ carboxylic alcohols, polyfunctional alcohols comprising 2 to 8 hydroxyl groups, alcohol ethers and polyalkylene glycols.

9. A composition according to claim 8, wherein said polyfunctional alcohols comprising 2 to 8 hydroxyl groups are chosen from ethylene glycol, hexylene glycol, glycerol and 1,2,6-hexanetriol.

10. A composition according to claim 8, wherein said alcohol ethers are chosen from methoxymethanol and ethoxyethanol.

11. A composition according to claim 3, wherein said at least one monomer of formula (Ia) is chosen from N,N-dialkylaminoalkyl acrylates, N,N-dialkylaminoalkyl methacrylates, N-dialkylaminoalkyl acrylamides and N-dialkylaminoalkyl methacrylamides, wherein the amide group may optionally be unsubstituted, N-alkyl-monosubstituted, N-alkylamino-monosubstituted or N,N-dialkylamino-disubstituted, and wherein the alkyl moities are chosen from linear C₁ to C₄₀ alkyl moities and branched C₃ to C₄₀ alkyl moities.

12. A composition according to claim 1, wherein said at least one ethylenically unsaturated monomer (a) is chosen from C₁ to C₄₀ vinyl esters, C₁ to C₄₀ alkyl esters, linear C₃ to C₄₀ carboxylic acids, branched C₃ to C₄₀ carboxylic acids, vinyl halides, allyl halides, vinyllactams, heterocyclic compounds substituted with at least one group chosen from vinyl groups and allyl groups, N-vinylimidazoles, diallylamines, vinylidene chloride, carbon-based unsaturated compounds, acrylic acid compounds quaternized with epichlorohydrin and methacrylic acid compounds quaternized with epichlorohydrin.

13. A composition according to claim 12, wherein said vinyllactams are chosen from vinylpyrrolidone and vinylcaprolactam.

14. A composition according to claim 12, wherein said heterocyclic compounds substituted with at least one group chosen from vinyl groups and allyl groups are chosen from vinylpyridine, vinyloxazoline and allylpyridine.

15. A composition according to claim 12, wherein said carbon-based unsaturated compounds are chosen from styrene and isoprene.

16. A composition according to claim 1, wherein said at least one ethylenically unsaturated monomer (a) is chosen from N-vinylimidazoles, diallylamines, vinylidene chloride, carbon-based unsaturated compounds, acrylic acid compounds quaternized with epichlorohydrin and methacrylic acid compounds quaternized with epichlorohydrin.

17. A composition according to claim 1, wherein said at least one ethylenically unsaturated monomers (a) is chosen from acrylic acid, methacrylic acid, ethacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate, n-butyl methacrylate, isobutyl methacrylate, t-butyl methacrylate, 2-ethylhexyl methacrylate, decyl methacrylate, methyl ethacrylate, ethyl ethacrylate, propyl ethacrylate, n-butyl ethacrylate, isobutyl ethacrylate, t-butyl ethacrylate, 2-ethylhexyl ethacrylate, decyl ethacrylate, 2,3-hydroxypropyl acrylate, 2,3-dihydroxypropyl methacrylate, 2-dihydroxyethyl acrylate, hydroxypropyl acrylate, 2-hydroxyethyl methacrylate, 2-hydroxyethyl ethacrylate, 2-methoxyethyl acrylate, 2-ethoxyethyl methacrylate, 2-ethoxyethyl ethacrylate, hydroxypropyl methacrylate, glyceryl monoacrylate, glyceryl monomethacrylate, polyalkylene glycol (meth)acrylates, unsaturated sulphonic acids, acrylamide, methacrylamide, ethacrylamide, N,N-dimethylacrylamide, N-ethylacrylamide, N-ethylmethacrylamide, 1-vinyl-imidazole, N,N-dimethylaminoethyl (meth)acrylate, maleic acid, fumaric acid, maleic anhydride, monoesters; of maleic anhydride, crotonic acid, itaconic acid, vinyl ethers, vinylformamide, vinylamine, vinylpyridine, vinylimidazole, vinylfuran, styrene, styryl sulphonate and allyl alcohol.

18. A composition according to claim 1, wherein said at least one ethylenically unsaturated monomer (a) further comprises at least one entity chosen from silicon atoms, fluorine atoms and thio groups.

19. A composition according to claim 1, wherein said at least one silicone compound (b) is chosen from at least one derivative of formula I: 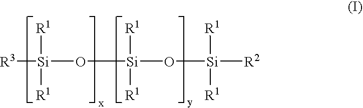
in which:
x and y, which may be identical or different, are each chosen from integers wherein said integers are chosen such that the number average molecular weight of said at least one silicone/acrylate copolymer ranges from 300 to 30,000;
R₂ and R₃, which may be identical or different, are each chosen from CH₃ and groups of formula: 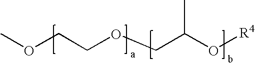
in which:
a and b, which may be identical or different, are each chosen from integers ranging from 0 to 50;
R⁴ is chosen from hydrogen, CH₃, groups of formula: 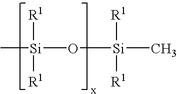
in which:
x is an integer chosen such that the number average molecular weight of said at least one silicone/acrylate copolymer ranges from 300 to 30,000; and
R¹, which may be identical or different, are each chosen from C₁ to C₂₀ aliphatic hydrocarbons, C₃ to C₂₀ aromatic groups, C₃ to C₂₀ cycloaliphatic hydrocarbons, groups comprising both aromatic groups and aliphatic groups and groups of formula: 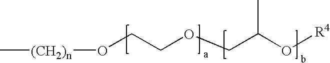
in which:
n is an integer ranging from 1 to 6;
a and b, which may be identical or different, are each chosen from integers ranging from 0 to 50; and
R⁴ is defined as above; and groups of formula: 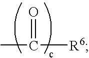
in which:
R⁶ is chosen from a C₁ to C₄₀ group, optionally comprising at least one group chosen from amino groups, carboxyl groups and sulfonyl groups, and, if c is equal to zero, R₆ is chosen from an anion of an inorganic acid; and
c is equal to 0 or 1; and
R¹, which may be identical or different, are each chosen from C1 to C20 aliphatic hydrocarbons, C₃ to C₂₀ aromatic groups, C₃ to C₂₀ cycloaliphatic hydrocarbons, groups comprising both aromatic groups and aliphatic groups and groups of formula: 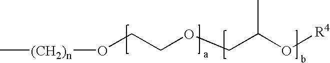
in which:
n is an integer ranging from 1 to 6;
a and b, which may be identical or different, are each chosen from integers ranging from 0 to 50; and
R⁴ is defined as above;
with the proviso that said at least one silicone derivative (b) comprises at least one oxyalkylene unit.

20. A composition according to claim 19, wherein said R¹, which may be identical or different, are each chosen from methyl groups, ethyl groups, propyl groups, butyl groups, isobutyl groups, pentyl groups, isopentyl groups, hexyl groups, octyl groups, decyl groups, dodecyl groups, octadecyl groups, cycloaliphatic groups, aromatic groups and groups comprising both aromatic and aliphatic groups.

21. A composition according to claim 20, wherein said cycloaliphatic groups are chosen from cyclohexyl groups.

22. A composition according to claim 20, wherein said aromatic groups are chosen from phenyl groups and naphthyl groups.

23. A composition according to claim 20, wherein said groups comprising both aromatic and aliphatic groups are chosen from benzyl groups, phenylethyl groups, tolyl groups and xylyl groups.

24. A composition according to claim 19, wherein said R⁴ is chosen from groups of formula —(CO)_{c}—R⁶, wherein c is equal to 1 and R⁶ is chosen from a group comprising from 1 to 40 carbon atoms, optionally comprising at least one group chosen from NH₂ groups, COOH groups and SO₃H groups, wherein said group comprising from 1 to 40 carbon atoms is chosen from an alkyl group, a cycloalkyl group and an aryl group.

25. A composition according to claim 19, wherein said R⁴ is chosen from groups of formula —(CO)_{c}, —R⁶, wherein c is equal to zero and R⁶ is chosen from phosphates and sulphates.

26. A composition according to claim 1, wherein said at least one silicone compound (b) is chosen from at least one derivative of formula: 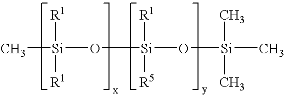
in which:
x and y, which may be identical or different, are each chosen from integers wherein said integers are chosen such that the number average molecular weight of said at least one silicone/acrylate copolymer ranges from 300 to 30,000;
R¹, which may be identical or different, are each chosen from C₁ to C₂₀ aliphatic hydrocarbons, C₃ to C₂₀ aromatic groups, C₃ to C₂₀ cycloaliphatic hydrocarbons, groups comprising both aromatic groups and aliphatic groups and groups of formula: 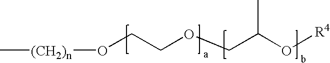
in which:
n is an integer ranging from 1 to 6;
a and b, which may be identical or different, are each chosen from integers ranging from 0 to 50; and
R⁴ is chosen from hydrogen, CH₃, groups of formula: 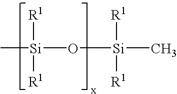
in which:
x is an integer chosen such that the number average molecular weight of said at least one silicone/acrylate copolymer ranges from 300 to 30,000; and
R¹, which may be identical or different, are each chosen from C₁ to C20 aliphatic hydrocarbons, C₃ to C₂₀ aromatic groups, C₃ to C₂₀ cycloaliphatic hydrocarbons, groups comprising both aromatic groups and aliphatic groups and groups of formula: 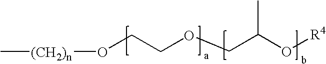
in which:
n is an integer ranging from 1 to 6;
a and b, which may be identical or different, are each chosen from integers ranging from 0 to 50; and
R⁴ is defined as above; and
groups of formula: 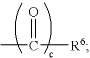
in which:
R⁶ is chosen from a C₁ to C₄₀ group, optionally comprising at least one group chosen from amino groups, carboxyl groups and sulfonyl groups, and, if c is equal to zero, R⁶ is chosen from an anion of an inorganic acid; and
c is equal to 0 or 1; and
R⁵, which may be identical or different, are each chosen from groups of formula: 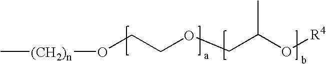
in which:
n is an integer ranging from 1 to 6;
a and b, which may be identical or different, are each chosen from integers ranging from 0 to 50; and
R⁴ is defined as above;
with the proviso that said at least one silicone derivative (b) comprises at least one oxyalkylene unit.

27. A composition according to claim 1, wherein said at least one silicone compound (b) is chosen from dimethicone copolyols and silicone surfactants.

28. A composition according to claim 1, wherein said at least one silicone compound (b) is present in a proportion ranging from 0.1% to 50% by weight relative to the total weight of said at least one silicone/acrylate copolymer.

29. A composition according to claim 28, wherein said at least one silicone compound (b) is present in a proportion ranging from 1% to 20% by weight relative to the total weight of said at least one silicone/acrylate copolymer.

30. A composition according to claim 1, wherein said at least one silicone/acrylate copolymer is chosen from water-soluble silicone/acrylate copolymers and silicone/acrylate copolymers having a dispersibility in water is such that, in a water/ethanol mixture dosed at 50/50 by volume, said copolymers are soluble in a proportion of greater than 0.1 g/l.

31. A composition according to claim 30, wherein said silicone/acrylate copolymers have a dispersibility in water such that said copolymers are soluble in a proportion of greater than 0.2 g/l.

32. A composition according to claim 1, wherein said at least one silicone/acrylate copolymer is present in a proportion ranging from 0.1% to 20% by weight relative to the total weight of said composition.

33. A composition according to claim 32, wherein said at least one silicone/acrylate copolymer is present in a proportion ranging from 0.5% to 10% by weight relative to the total weight of said composition.

34-37. (canceled)

38. A composition according to claim 1, wherein said at least one non-ionic polymer comprising at least one vinyllactam unit has a number-average molecular mass greater than about 5000.

39. A composition according to claim 38, wherein said number-average molecular mass ranges from about 10,000 to about 1,000,000.

40. A composition according to claim 38, wherein said number-average molecular mass ranges from about 10,000 to about 100,000.

41. A composition according to claim 1, wherein said at least one nonionic polymer comprising at least one vinyllactam unit is present in a proportion ranging from 0.1% to 10% by weight relative to the total weight of said at least one silicone/acrylate copolymer.

42. A composition according to claim 41, wherein said at least one nonionic polymer comprising at least one vinyllactam unit is present in a proportion ranging from 0.2% to 5% by weight relative to the total weight of said at least one silicone/acrylate copolymer.

43. A composition according to claim 2, wherein said at least one cosmetically acceptable medium is chosen from water and cosmetically acceptable solvents.

44. A composition according to claim 43, wherein said cosmetically acceptable solvents are chosen from alcohols and mixtures comprising water and at least one solvent.

45. A composition according to claim 44, wherein said at least one solvent is chosen from C₁-C₄ alcohols.

46. A composition according to claim 45, wherein said C₁-C₄ alcohol is chosen from ethanol and isopropanol.

47. A composition according to claim 46, wherein said C₁-C₄ alcohol is chosen from ethanol.

48. A composition according to claim 1 further comprising at least one suitable additive chosen from anionic surfactants, cationic surfactants, nonionic surfactants, amphoteric surfactants, fragrances, screening agents, preserving agents, proteins, vitamins, polymers different from said at least one silicone/acrylate copolymer and different from said at least one nonionic polymer defined in claim 1, plant oils, mineral oils and synthetic oils.

49. A composition according to claim 1, wherein said composition is a cosmetic composition.

50. A process for holding or shaping a hairstyle, comprising applying to hair an effective amount of a composition comprising:
(a) at least one silicone/acrylate copolymer; and
(b) at least one nonionic polymer comprising at least one vinyllactam unit chosen from polyvinylpyrrolidone/vinyl acetate/vinyl proprionate terpolymers,
wherein said at least one silicone/acrylate copolymer is derived from radical-mediated polymerization of:
(i) at least one ethylenically unsaturated monomer (a); and
(ii) at least one silicone compound (b) comprising at least one oxyalklyene group.

51. A process according to claim 50, wherein said composition further comprises a cosmetically acceptable medium.

52. A process according to claim 50, wherein said at least one ethylenically unsaturated monomer (a) is chosen from at least one monomer of formula (Ia): 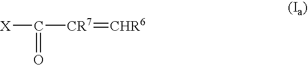
in which:
X is chosen from OH, OM, OR⁸, NH₂, NHR⁸ and N(R⁸)₂, wherein:
R⁸, which may be identical or different, are each chosen from hydrogen atoms, linear and branched C₁ to C₄₀ alkyl groups, optionally substituted with at least one group chosen from alkoxy groups, amino groups and carboxyl groups, monohydroxylated linear and branched C₁ to C₄₀ alkyl groups, optionally substituted with at least one group chosen from alkoxy groups, amino groups and carboxyl groups, polyhydroxylated linear and branched C₁ to C₄₀ alkyl groups, optionally substituted with at least one group chosen from alkoxy groups, amino groups and carboxyl groups, and hydroxylated polyethers; and
M is chosen from Na⁺, K⁺, Mg⁺⁺, NH⁴⁺, an alkylammonium group, a dialkylammonium group, a trialkylammonium group and a tetraalkylammonium group;
R⁷ and R⁶, which may be identical or different, are each chosen from hydrogen atoms, linear and branched C₁ to C₈ alkyl groups, methoxy groups, ethoxy groups, 2-hydroxyethoxy groups, 2-methoxyethoxy groups, 2-ethoxyethyl groups, CN groups, COOH groups and COOM groups, wherein M is defined as above.

53. A process according to claim 52, wherein said linear and branched C₁ to C₄₀ alkyl groups optionally substituted with at least one group chosen from alkoxy groups, amino groups and carboxyl groups are each chosen from N,N-dimethylaminoethyl groups, 2-methoxyethyl groups, 2-ethoxyethyl groups, methoxypropyl groups and ethoxypropyl groups.

54. A process according to claim 52, wherein said monohydroxylated linear and branched C₁ to C₄₀ alkyl groups optionally substituted with at least one group chosen from alkoxy groups, amino groups and carboxyl groups are each chosen from 2-hydroxyethyl groups and hydroxypropyl groups.

55. A process according to claim 52, wherein said at least one monomer of formula (Ia) is chosen from acrylic acid, the salts of acrylic acid, esters and amides.

56. A process according to claim 52, wherein said at least one monomer of formula (Ia) is chosen from methacrylic acid, ethacrylic acid and 3-cyanoacrylic acid.

57. A composition according to claim 52, wherein said at least one monomer of formula (Ia) is chosen from esters obtained from linear C₁ to C₄₀ alkyls, branched C₃ to C₄₀ alkyls, of C₃ to C₄₀ carboxylic alcohols, polyfunctional alcohols comprising 2 to 8 hydroxyl groups, alcohol ethers and polyalkylene glycols.

58. A process according to claim 57, wherein said polyfunctional alcohols comprising 2 to 8 hydroxyl groups are chosen from ethylene glycol, hexylene glycol, glycerol and 1,2,6-hexanetriol.

59. A process according to claim 57, wherein said alcohol ethers are chosen from methoxymethanol and ethoxyethanol.

60. A process according to claim 52, wherein said at least one monomer of formula (Ia) is chosen from N,N-dialkylaminoalkyl acrylates, N,N-dialkylaminoalkyl methacrylates, N-dialkylaminoalkyl acrylamides and N-dialkylaminoalkyl methacrylamides, wherein the amide group may optionally be unsubstituted, N-alkyl-monosubstituted, N-alkylamino-monosubstituted or N,N-dialkylamino-disubstituted, and wherein the alkyl moities are chosen from linear C₁ to C₄₀ alkyl moities and branched C₃ to C₄₀ alkyl moities.

61. A process according to claim 50, wherein said at least one ethylenically unsaturated monomer (a) is chosen from C₁ to C₄₀ vinyl esters, C₁ to C₄₀ alkyl esters, linear C₃ to C₄₀ carboxylic acids, branched C₃ to C₄₀ carboxylic acids, vinyl halides, allyl halides, vinyllactams, heterocyclic compounds substituted with at least one group chosen from vinyl groups and allyl groups, N-vinylimidazoles, diallylamines, vinylidene chloride, carbon-based unsaturated compounds, acrylic acid compounds quaternized with epichlorohydrin and methacrylic acid compounds quaternized with epichlorohydrin.

62. A process according to claim 61, wherein said vinyllactams are chosen from vinylpyrrolidone and vinylcaprolactam.

63. A process according to claim 61, wherein said heterocyclic compounds substituted with at least one group chosen from vinyl groups and allyl groups are chosen from vinylpyridine, vinyioxazoline and allylpyridine.

64. A process according to claim 61, wherein said carbon-based unsaturated compounds are chosen from styrene and isoprene.

65. A composition according to claim 50, wherein said at least one ethylenically unsaturated monomer (a) is chosen from N-vinylimidazoles, diallylamines, vinylidene chloride, carbon-based unsaturated compounds, acrylic acid compounds quaternized with epichlorohydrin and methacrylic acid compounds quaternized with epichlorohydrin.

66. A process according to claim 50, wherein said at least one ethylenically unsaturated monomers (a) is chosen from acrylic acid, methacrylic acid, ethacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate, n-butyl methacrylate, isobutyl methacrylate, t-butyl methacrylate, 2-ethylhexyl methacrylate, decyl methacrylate, methyl ethacrylate, ethyl ethacrylate, propyl ethacrylate, n-butyl ethacrylate, isobutyl ethacrylate, t-butyl ethacrylate, 2-ethylhexyl ethacrylate, decyl ethacrylate, 2,3-hydroxypropyl acrylate, 2,3-dihydroxypropyl methacrylate, 2-dihydroxyethyl acrylate, hydroxypropyl acrylate, 2-hydroxyethyl methacrylate, 2-hydroxyethyl ethacrylate, 2-methoxyethyl acrylate, 2-ethoxyethyl methacrylate, 2-ethoxyethyl ethacrylate, hydroxypropyl methacrylate, glyceryl monoacrylate, glyceryl monomethacrylate, polyalkylene glycol (meth)acrylates, unsaturated sulphonic acids, acrylamide, methacrylamide, ethacrylamide, N,N-dimethylacrylamide, N-ethylacrylamide, N-ethyl methacrylamide, 1-vinyl-imidazole, N,N-dimethylaminoethyl (meth)acrylate, maleic acid, fumaric acid, maleic anhydride, monoesters of maleic anhydride, crotonic acid, itaconic acid, vinyl ethers, vinylformamide, vinylamine, vinylpyridine, vinylimidazole, vinylfuran, styrene, styryl sulphonate and allyl alcohol.

67. A process according to claim 50, wherein said at least one ethylenically unsaturated monomer (a) further comprises at least one entity chosen from silicon atoms, fluorine atoms and thio groups.

68. A process according to claim 50, wherein said at least one silicone compound (b) is chosen from at least one derivative of formula I: 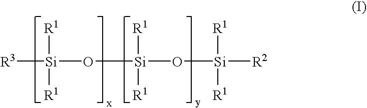
in which:
x and y, which may be identical or different, are each chosen from integers wherein said integers are chosen such that the number average molecular weight of said at least one silicone/acrylate copolymer ranges from 300 to 30,000;
R² and R³, which may be identical or different, are each chosen from CH₃ and groups of formula: 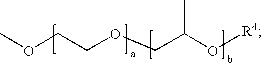
in which:
a and b, which may be identical or different, are each chosen from integers ranging from 0 to 50;
R⁴ is chosen from hydrogen, CH₃, groups of formula: 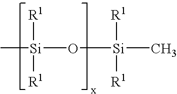
in which:
x is an integer chosen such that the number average molecular weight of said at least one silicone/acrylate copolymer ranges from 300 to 30,000; and
R¹, which may be identical or different, are each chosen from C₁ to C₂₀ aliphatic hydrocarbons, C₃ to C₂₀ aromatic groups, C₃ to C₂₀ cycloaliphatic hydrocarbons, groups comprising both aromatic groups and aliphatic groups and groups of formula: 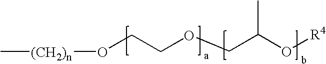
in which:
n is an integer ranging from 1 to 6;
a and b, which may be identical or different, are each chosen from integers ranging from 0 to 50; and
R⁴ is defined as above; and
groups of formula: 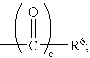
in which:
R⁶ is chosen from a C₁ to C₄₀ group, optionally comprising at least one group chosen from amino groups, carboxyl groups and sulfonyl groups, and, if c is equal to zero, R6 is chosen from an anion of an inorganic acid; and
c is equal to 0 or 1; and
R¹, which may be identical or different, are each chosen from C₁ to C₂₀ aliphatic hydrocarbons, C₃ to C₂₀ aromatic groups, C₃ to C₂₀ cycloaliphatic hydrocarbons, groups comprising both aromatic groups and aliphatic groups and groups of formula: 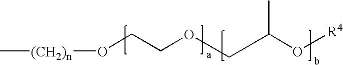
in which:
n is an integer ranging from 1 to 6;
a and b, which may be identical or different, are each chosen from integers ranging from 0 to 50; and
R⁴ is defined as above;
with the proviso that said at least one silicone derivative (b) comprises at least one oxyalkylene unit.

69. A process according to claim 68, wherein said R¹, which may be identical or different, are each chosen from methyl groups, ethyl groups, propyl groups, butyl groups, isobutyl groups, pentyl groups, isopentyl groups, hexyl groups, octyl groups, decyl groups, dodecyl groups, octadecyl groups, cycloaliphatic groups, aromatic groups and groups comprising both aromatic and aliphatic groups.

70. A process according to claim 69, wherein said cycloaliphatic groups are chosen from cyclohexyl groups.

71. A process according to claim 69, wherein said aromatic groups are chosen from phenyl groups and naphthyl groups.

72. A process according to claim 69, wherein said groups comprising both aromatic and aliphatic groups are chosen from benzyl groups, phenylethyl groups, tolyl groups and xylyl groups.

73. A process according to claim 68, wherein said R⁴ is chosen from groups of formula —(CO)_{c}—R⁶, wherein c is equal to 1 and R⁶ is chosen from a group comprising from 1 to 40 carbon atoms, optionally comprising at least one group chosen from NH2 groups, COOH groups and SO₃H groups, wherein said group comprising from 1 to 40 carbon atoms is chosen from an alkyl group, a cycloalkyl group and an aryl group.

74. A process according to claim 68, wherein said R⁴ is chosen from groups of formula —(CO)_{c}—R⁶, wherein c is equal to zero and R⁶ is chosen from phosphates and sulphates.

75. A process according to claim 50, wherein said at least one silicone compound (b) is chosen from at least one derivative of formula: 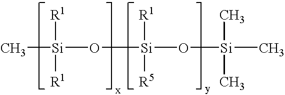
in which:
x and y, which may be identical or different, are each chosen from integers wherein said integers are chosen such that the number average molecular weight of said at least one silicone/acrylate copolymer ranges from 300 to 30,000;
R¹, which may be identical or different, are each chosen from C₁ to C₂ aliphatic hydrocarbons, C₃ to C₂₀ aromatic groups, C₃ to C₂₀ cycloaliphatic hydrocarbons, groups comprising both aromatic groups and aliphatic groups and groups of formula: 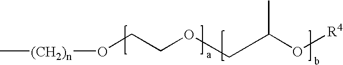
in which:
n is an integer ranging from 1 to 6;
a and b, which may be identical or different, are each chosen from integers ranging from 0 to 50; and
R⁴ is chosen from hydrogen, CH₃, groups of formula: 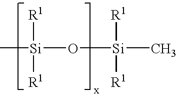
in which:
x is an integer chosen such that the number average molecular weight of said at least one silicone/acrylate copolymer ranges from 300 to 30,000; and
R¹, which may be identical or different, are each chosen from C₁ to C₂₀ aliphatic hydrocarbons, C₃ to C₂₀ aromatic groups, C₃ to C₂₀ cycloaliphatic hydrocarbons, groups comprising both aromatic groups and aliphatic groups and groups of formula: 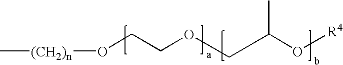
in which:
n is an integer ranging from 1 to 6;
a and b, which may be identical or different, are each chosen from integers ranging from 0 to 50; and
R⁴ is defined as above; and
groups of formula: 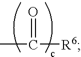
in which:
R⁶ is chosen from a C₁ to C₄₀ group, optionally comprising at least one group chosen from amino groups, carboxyl groups and sulfonyl groups, and, if c is equal to zero, R₆ is chosen from an anion of an inorganic acid; and
c is equal to 0 or 1; and
R⁵, which may be identical or different, are each chosen from groups of formula: 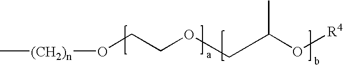
in which:
n is an integer ranging from 1 to 6;
a and b, which may be identical or different, are each chosen from integers ranging from 0 to 50; and
R⁴ is defined as above;
with the proviso that said at least one silicone derivative (b) comprises at least one oxyalkylene unit.

76. A process according to claim 50, wherein said at least one silicone compound (b) is chosen from dimethicone copolyols and silicone surfactants.

77. A process according to claim 50, wherein said at least one silicone compound (b) is present in a proportion ranging from 0.1% to 50% by weight relative to the total weight of said at least one silicone/acrylate copolymer.

78. A process according to claim 77, wherein said at least one silicone compound (b) is present in a proportion ranging from 1% to 20% by weight relative to the total weight of said at least one silicone/acrylate copolymer.

79. A process according to claim 50, wherein said at least one silicone/acrylate copolymer is chosen from water-soluble silicone/acrylate copolymers and silicone/acrylate copolymers having a dispersibility in water is such that, in a water/ethanol mixture dosed at 50/50 by volume, said copolymers are soluble in a proportion of greater than 0.1 g/l.

80. A process according to claim 79, wherein said silicone/acrylate copolymers have a dispersibility in water such that said copolymers are soluble in a proportion of greater than 0.2 g/l.

81. A process according to claim 50, wherein said at least one silicone/acrylate copolymer is present in a proportion ranging from 0.1% to 20% by weight relative to the total weight of said composition.

82. A process according to claim 81, wherein said at least one silicone/acrylate copolymer is present in a proportion ranging from 0.5% to 10% by weight relative to the total weight of said composition.

83-86. (canceled)

87. A process according to claim 50, wherein said at least one nonionic polymer comprising at least one vinyllactam unit has a number-average molecular mass greater than about 5000.

88. A process according to claim 87, wherein said number-average molecular mass ranges from about 10,000 to about 1,000,000.

89. A process according to claim 88 wherein said number-average molecular mass ranges from about 10,000 to about 100,000.

90. A process according to claim 50, wherein said at least one nonionic polymer comprising at least one vinyllactam unit is present in a proportion ranging from 0.1% to 10% by weight relative to the total weight of said at least one silicone/acrylate copolymer.

91. A process according to claim 90, wherein said at least one nonionic polymer comprising at least one vinyllactam unit is present in a proportion ranging from 0.2% to 5% by weight relative to the total weight of said at least one silicone/acrylate copolymer.

92. A process according to claim 51, wherein said at least one cosmetically acceptable medium is chosen from water and cosmetically acceptable solvents.

93. A process according to claim 92, wherein said cosmetically acceptable solvents are chosen from alcohols and mixtures comprising water and at least one solvent.

94. A process according to claim 93, wherein said at least one solvent is chosen from C₁-C₄ alcohols.

95. A process according to claim 94, wherein said C₁-C₄ alcohol is chosen from ethanol and isopropanol.

96. A process according to claim 95, wherein said C₁-C₄ alcohol is chosen from ethanol.

97. A process according to claim 50 further comprising at least one suitable additive chosen from anionic surfactants, cationic surfactants, nonionic surfactants, amphoteric surfactants, fragrances, screening agents, preserving agents, proteins, vitamins, polymers different from said at least one silicone/acrylate copolymer and different from said at least one nonionic polymer defined in claim 50, plant oils, mineral oils and synthetic oils.

98. A process for making a cosmetic product comprising including in said product at least one composition comprising:
(a) at least one silicone/acrylate copolymer; and
(b) at least one nonionic polymer comprising at least one vinyllactam unit chosen from polyvinylpyrrolidone/vinyl acetate/vinyl proprionate terpolymers,
wherein said at least one silicone/acrylate copolymer is derived from radical-mediated polymerization of:
(i) at least one ethylenically unsaturated monomer (a); and
(ii) at least one silicone compound (b) comprising at least one oxyalkylene group.

99. A process according to claim 98, wherein said cosmetic product is a hair product.

100. A process according to claim 99, wherein said hair product holds and shapes a hairstyle.

101. A process according to claim 98, wherein said cosmetic product is a product for skin, a product for nails, a product for lips, a product for hair, a product for eyebrows or a product for eyelashes.

102. A product for skin, a product for nails, a product for lips, a product for hair, a product for eyebrows or a product for eyelashes comprising:
(a) at least one silicone/acrylate copolymer; and
(b) at least one nonionic polymer comprising at least one vinyllactam unit chosen from polyvinylpyrrolidone/vinyl acetate/vinyl proprionate terpolymers,
wherein said at least one silicone/acrylate copolymer is derived from radical-mediated polymerization of:
(i) at least one ethylenically unsaturated monomer (a); and
(ii) at least one silicone compound (b) comprising at least one oxyalkylene group.
